(19) United States
(12) Patent Application Publication
(10) Pub. No.: US 2022/0218863 A1
(43) Pub. Date: Jul. 14, 2022

(54) SYSTEMS, METHODS, AND ARTICLES FOR DELIVERY OF SUBSTANCES IN VESSELS

(71) Applicant: SENSORY CLOUD, INC., Boston, MA (US)

(72) Inventors: David A. Edwards, Cambridge, MA (US); Rachel Diane Field, Huntington Beach, CA (US); Tom Devlin, Somerville, MA (US)

(21) Appl. No.: 17/606,201

(22) PCT Filed: Apr. 24, 2020
(86) PCT No.: PCT/US2020/029874

§ 371 Date: Oct. 25, 2021

Related U.S. Application Data
(60) Provisional application No. 62/838,740, filed on Apr. 25, 2019.

(51) Int. Cl. A61L 9/14; A61M 15/08; A61M 11/00

(52) U.S. Cl. CPC A61L 9/14; A61M 15/08; A61M 11/005; A61M 2205/3386; A61L 2209/111; A61L 2209/134; A61M 2205/18; A61L 2209/132

(57) ABSTRACT

A system to provide media (e.g., scent, therapeutics, other substances) includes a dispenser that may include a reservoir, an actuator (e.g., nebulizer), and an outlet through which a vapor or aerosol may be supplied or dispensed, the vapor or aerosol, for example comprising readily-soluble droplets having a median size range of approximately 2 microns to approximately 50, 20, or 10 microns for introduction to vessel. An interface is positionable over an opening of the vessel to close the opening while allow media to be dispensed into the interior of the vessel, thereby retaining the dispensed media until taken in by a user. The interface (e.g., annular plate) has a pair of opposed major surfaces, and the first major face supported on a rim of the vessel, with the dispenser supported on the second major face in an inverted orientation. A sensor may sense orientation, and cause an actuator (e.g., nebulizer) to dispense media when in the inverted orientation or otherwise titled for normal.

### BACKGROUND

### Field

This disclosure generally relates to providing substances (e.g., scent, therapeutic, and/or other substances) via a dispenser and a vessel, for instance in a form (e.g., vapor, aerosol) suitable for retro-nasal delivery, which may be used to modulate human or other animal biological processes or treat medical conditions, and in particular to related compositions, systems, methods, and articles of manufacture.

### Description of the Related Art

All of our five sense act as messengers that deliver information to the brain, which then processes this information, causing us to respond in relatively predictable ways. Within the context of our sense of smell, all odors present themselves in specific chemical configurations, allowing humans to perceive a wide variety of distinct odors. Odor perception initiates in the nose, where the respective molecules are detected by a large family of olfactory receptors. Olfactory receptors have diverse protein sequences, and are assigned to subfamilies on the basis of sequence relationships. These observations formed the basis for research into the mechanisms underlying human odor perception, leading to the 2004 grant of the Nobel Prize in Physiology and Medicine to Linda B. Buck and Richard Axel.

However, even given the significant importance of our sense of smell, relatively little has been done to develop the apparent physiological value of this sense or to more thoroughly incorporate it into how humans experience the world around them on a daily basis. Although some systems and devices have been proposed for attempting to provide olfactory sensations to users (see, for instance, U.S. Pat. Nos. 8,050,545, 8,032,014, 6,654,664 and 6,803,987), they have proven inadequate as mobile, personal, targeted and effective delivery systems that may be used to alter behavior.

As explained herein, new approaches that effectively deliver therapeutic and other substances in order to elicit a physiological response are desirable.

### BRIEF SUMMARY

Recent advances in olfaction biology have made it clear that flavor images that appear in the brain as a consequence of activating sensory receptors in the process of eating and drinking play a role in up- and down-regulating of metabolic function. Among the most important of sensory receptors involved in the creation of these flavor images are olfactory receptors in the nasal epithelium. Such receptors appear elsewhere in the body, including the heart, gut, and circulating cells of the immune system. As such, stimulation of olfactory receptors can influence not only metabolic processes but other processes including those involved in immunity and brain function.

The delivery of substances (i.e., active substances) to the nasal epithelium to modulate human health, as in the delivery of active substances for relieving congestion, or symptoms related to asthma, generally involves the delivery of dry or liquid formulations to the nose via a nebulizer, metered dose inhaler, or dry powder inhaler. These delivery modalities conventionally involve spraying or sniffing active substances directly into the nose via the nostrils or nasal vestibule.

Active substances deposit in the nose, depending on the nature of the delivery system and technique, with some associated degree of efficiency. This efficiency can be measured as a fraction of “delivered dose” to “nominal dose.” Delivered dose is the mass of active substance that not only deposits on the nasal epithelium, but is delivered to the target tissues and/or receptors. Given that clearance of the active substance from the nose is rapid, delivery to the nose of the dose of active substance in a form that is quickly dissolved and distributed is highly desirable.

Naturally, delivery of odorants (i.e., scent molecules) to the nasal epithelium occurs in two ways. The first, ortho-nasal scent delivery, occurs by sniffing odorants in the atmosphere, e.g., directly via the nostrils or nasal vestibule. The second, retro-nasal scent delivery, occurs by the natural diffusion and convection of odorants in the mouth into the nasal passages via the oropharynx. This latter delivery is referred to as retro-nasal olfaction, and is promoted by exhalation.

It has recently been found that many people who cannot perceive scent via ortho-nasal olfaction, can actually perceive scent or flavor via retro-nasal olfaction. The surprising “special capacity” of retro-nasal olfaction relates to the fact that the human oropharynx is supremely well designed to bring odorants in the mouth into the nasal passages. As a consequence, flavor perception plays a critical role in the regulation of human metabolism. Humans develop likes and cravings for certain foods as a consequence of experiencing the metabolic effects of these foods, and associating these effects with flavor images in their brains. Eventually, these images, as memories (the olfactory nerve links olfactory receptors in the nose with the seat of long-term memory, the hippocampus) drive food interests and cravings that lead to humans receiving the metabolic effects they enjoy.

Recently, human and animal studies have found that simply perceiving the scent of certain foods, like chocolate or the aroma of roasted coffee beans, can trigger metabolic effects that heretofore have been believed to occur only on the ingestion of chocolate or coffee. This surprising finding, combined with the discovery of the general efficacy of retro-nasal olfaction versus ortho-nasal olfaction, opens up a completely new opportunity for active substance (e.g., drugs, and various scent molecules that have until now principally been understood to relate to food and flavor perception) delivery to the nose.

Described herein are new compositions, apparatus, methods and articles for delivery of active substances via the nose. The described compositions, apparatus, methods and articles can be employed for the up- and down-regulation of human (and other animal) metabolism, as well as to other beneficial physiological effects, for instance decongestion. Rather than deliver active substances to the nose via the standard ortho-nasal route, the described approaches advantageously deliver active substances to the nose via the retro-nasal route. The active substances are formulated in readily-soluble water droplets that have a median size range of 2-50 microns, 2-20 microns, or 2-10 microns, advantageously too large for significant penetration into the lungs, while small enough to be carried into the nose.

As also described herein, apparatus are provided which allow the portable, discrete delivery of active substances, enhancing or efficiency of delivery to humans and other animals. Advantageously, the apparatus is configured to be portable, allowing the user to have the benefit of retro-nasal delivery, on demand, in a wide variety of environments. The apparatus and compositions may be used to enhance the efficiency of delivery of active substances, and to provoke a physiological response in a human or other animal via the connection of the olfactory sensory system.

In some implementations, a device is provided in the form of a dispenser which is operable to emit media (e.g., scent, therapeutic, other substances) into a vessel (e.g., drinking glass, container), for example as a spray of droplets or aerosol. The vessel may have an interior that may in use hold an ingestible food item, for instance in a liquid (e.g., a beverage), solid, gel or colloidal suspension form. The dispenser includes a media reservoir that is distinct from the interior of the vessel, and which in use holds media (e.g., scent media, therapeutic media, other media). The dispenser is used to dispense substance or media is into the vessel, for example as a spray of droplets or aerosol containing one more scents or other therapeutics or other substances, for instance over a beverage or other editable item. Use of a dispenser with a media reservoir that is separate from the vessel advantageously allows creation or generation of clouds of flavor/scent above food and drink, the clouds which differ from the aroma of the drink or food itself, and can be useful for conditioning appetite.

An outlet (e.g., port, nozzle) of the dispenser may be positioned and oriented so that a spray or other distribution of scent media is directed towards and into the interior of the vessel. The vessel typically has an opening, from which the contents of the vessel may be sampled by a user and via which media can be dispensed into the interior of the vessel. An interface may be employed to provide an interface between the dispenser and the vessel, the interface having a shape and size to substantially close the opening of the vessel while allow providing a path to allow dispensing of media into the interior of the vessel by the device or dispenser, to advantageously ensure that the dispensed media at least initially stays in the interior of the vessel, for instance until sampled or “ingested” by a human end user.

In some implementations, the interface is a substrate having a first major face, a second major face opposed from the first major face across a thickness of the interface, and a through-hole that extends through the thickness of the substrate. The interface may, for example, take the form of an annular disc.

In some implementations, the interface is separate and distinct from the dispenser, the interface individually positionable on a top of the vessel with a first major face of the interface facing the vessel, and the dispenser individually positionable on a second major face of the interface, for example subsequent to positioning the interface with respect to the vessel. Providing the interface as a separate item from the dispenser may advantageously allow a set of interfaces to be provided, and selected based on a size or shape of the vessel or of the opening in the vessel. In some implementations, the interface is attached to the dispenser as an integral unit. In such implementations, the interface may be permanently fixed to a body of the dispenser, or removable detachably mounted to the body of the dispenser. Removable attachment may advantageously allow a set of interfaces to be provided, and selected based on a size or shape of the vessel or of the opening in the vessel. Attachment may be via magnets or fasteners.

In some implementations, the interface may be securable to the vessel. In such implementations, the interface may include an elongated hollow conduit (e.g., a straw), that provides a fluidly communicative path from an interior to the vessel to an exterior thereof, and which allows for a user or consumer to “sip out” or imbibe the “cloud” which is otherwise contained in the interior of the vessel.

The dispenser may include an actuator operable to transform media from one form to another, for example a nebulizer operable to generate a vapor or aerosol from the media in the reservoir. The nebulizer may, for example, include a screen and a piezo-electric element, solenoid, or an electric motor physically (e.g., mechanically, magnetically) coupled to move (e.g., oscillate, rotate) the screen and thereby cause dispersion of the media in the interior of the vessel, for instance as a spray or aerosol.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is an isometric view of a dispenser, interface and vessel according to at least one illustrated embodiment, the interface shown positioned over an opening of the vessel and the dispenser shown spaced from the interface and vessel.

FIG. 1B is an isometric view of the dispenser, interface and vessel of FIG. 1A, the interface shown positioned over the opening of the vessel and the dispenser shown positioned on the interface to dispense media into an interior of the vessel while the interface retains the media within the interior of the vessel.

FIG. 2A is an exploded view of a dispenser with an integral interface, the dispenser operable to deliver a vapor, a cloud, or an aerosol comprising media (e.g., scent media, therapeutic, other substances) into a vessel, according to at least one illustrated embodiment.

FIG. 2B is a perspective view of the dispenser of FIG. 2A.

FIG. 2C is a side view of the dispenser of FIGS. 2A and 2B.

FIG. 2D is a cross-sectional side view of the dispenser of FIGS. 2A-2B.

FIG. 2E is a side view of components of the dispenser of FIGS. 2A-2D.

FIG. 2F is another side view of components of the dispenser of FIGS. 2A-2D.

FIG. 3A is a rear view of a printed circuit board and associated components coupled thereto for use in the dispenser of FIGS. 2A-2F, according to at least one illustrated embodiment.

FIG. 3B is a side view of the printed circuit board and associated components of FIG. 3A.

FIG. 3C is a front view of the printed circuit board and associated components of FIGS. 3A and 3B.

FIG. 3D is a perspective view of the printed circuit board and associated components of FIGS. 3A-3C.

FIG. 3E is a rear view of the printed circuit board of FIGS. 3A-3D, without the associated components coupled thereto.

FIG. 3F is a side view of the printed circuit board of FIGS. 3A-3D, without the associated components coupled thereto.

FIG. 3G is a front view of the printed circuit board of FIGS. 3A-3D, without the associated components coupled thereto.

FIG. 3H is a front view of an alternative configuration of the printed circuit board of FIGS. 3A-3D, without the associated components coupled thereto.

FIG. 4 is a three-dimensional rendering in a perspective view of the dispenser and separate interface, operable to deliver a vapor, a cloud, or an aerosol to a vessel, according to at least one illustrated embodiment.

FIG. 5 is a three-dimensional rendering in a perspective view of an alternative configuration for the dispenser and separate interface, operable to deliver a vapor, a cloud, or an aerosol to a vessel, according to at least one illustrated embodiment.

FIG. 6 is a three-dimensional rendering in a perspective view of another alternative configuration for the dispenser and separate interface, operable to deliver a vapor, a cloud, or an aerosol to a vessel, according to at least one illustrated embodiment.

FIG. 7 is a three-dimensional rendering in a perspective view of another alternative configuration for the dispenser and separate interface, operable to deliver a vapor, a cloud, or an aerosol to a vessel, according to at least one illustrated embodiment.

FIG. 8 is a three-dimensional rendering in a perspective view of another alternative configuration for the dispenser and separate interface, operable to deliver a vapor, a cloud, or an aerosol to a vessel, according to at least one illustrated embodiment.

FIG. 9 is a three-dimensional rendering in a perspective view of another alternative configuration for the dispenser and separate interface, operable to deliver a vapor, a cloud, or an aerosol to a vessel, according to at least one illustrated embodiment.

FIG. 10 is a three-dimensional rendering in a perspective view of another alternative configuration for the dispenser and separate interface, operable to deliver a vapor, a cloud, or an aerosol to a vessel, according to at least one illustrated embodiment.

FIG. 11 is an isometric view of a portion of a dispenser illustrating a nebulizer which can include a screen and at least one of a piezo-electric element, solenoid or electric motor physically coupled to move the screen, the dispenser also including one or more of a radio, a transducer or sensor and a switch communicatively coupled to a control system, for example a microcontroller and memory, and operably coupled to control operation of the nebulizer, according to at least one illustrated implementation.

FIG. 12A is a side elevational view of a vessel with a cover positioned to close a top or opening of the vessel, a conduit or straw that provides a fluidly communicative path through the top, according to at least one illustrated implementation.

FIG. 12B is a side elevational view of a vessel with the cover removed to open the top or opening of the vessel, providing access to an interior thereof, a conduit or straw physically coupled to, or forming part of, the cover that provides a fluidly communicative path through the top.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with microcontrollers, piezo-electric devices, Peltier devices, power supplies such as DC/DC converters, wireless radios (i.e., transmitters, receivers or transceivers), computing systems including client and server computing systems, and networks (e.g., cellular, packet switched), as well as other communications channels, have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

Reference throughout this specification to “media” means scent media, fragrance media, and/or active media for example therapeutic media which can elicit of therapeutic effect when received by a human or other animal, for existence via retro-nasal delivery. Media may provide a sensory effect, for instance eliciting a pleasant sensation and/or elicit a therapeutic effect. Media may take a variety of forms including, for example, liquid, solid, powder, gel, colloidal suspension, and/or essential oils.

The headings and Abstract of the Disclosure provided herein are for convenience only and do not interpret the scope or meaning of the embodiments.

In particular, described herein are new compositions, systems, methods, and articles of manufacture to advantageously delivery of one or more active substances to the nose via retro-nasal delivery. Such can be employed in the up-regulation and, or, down-regulation of human and other animal metabolism. Such can additionally or alternatively be employed to produce other beneficial physiological effects, for example decongestion. Rather than deliver active substances to the nose via the standard ortho-nasal route, the compositions, apparatus, methods and articles described herein advantageously deliver active substances to the nose via the retro-nasal route.

These active substances or compositions are advantageously formulated as or in readily-soluble water droplets. The readily-soluble water droplets have a median size range of approximately 2 microns to approximately 50, 20, or 10 microns. Thus, the readily-soluble water droplets are too large for significant penetration into the lungs, while being small enough to be carried into the nose.

The active substances can be dissolved, if water soluble, directly in the water droplets. The active substances can, for example if not water soluble, be encapsulated inside, or otherwise formulated as, micelles, micro-emulsions, emulsions, liposomes, nanoparticles or other kinds of colloids. These colloids do not have a size larger than 500 nm, and optimally 200 nm or smaller. The small size of these colloids permits the nebulization of the droplets without destroying the colloids or otherwise impeding (e.g., clogging) the nebulizer (e.g., ultrasound transducer).

The droplets are delivered as a cloud to the mouth, for instance by the act of sipping. Sipping can either involve simply placing one's lips in a cloud of droplets containing the active material and sipping, or involve taking the droplets into the mouth via a conduit (e.g., a straw). On sipping the cloud, the droplets are delivered to the mouth where the droplets are suspended in the air in the mouth, and settle by gravity. The vapor around the droplets can immediately bring active substance into the nose via a “chimney effect” of the nose, and the droplets will themselves waft into the nose and deposit there, delivering active substances in a form that quickly acts on or within active tissue and resists quick clearance. Notably when the active substances are in small colloidal (e.g., nanoparticle) form, the colloids will themselves tend to resist clearance whereas larger particulates than those encompassed by the compositions described here will tend to be cleared through mucocilliary action. The active-substance-loaded droplets described here are produced from a small reservoir of less than 100 ML, optimally less than 50 ML, and particularly optimally less than 25 ML.

The composition, apparatus, methods and articles described herein have various useful benefits. The delivery of active substances or compositions (e.g., odorants or flavorful molecules of some kind, or other more traditional therapeutics) that up- and down-regulate metabolism, can be achieved with less than 1 gram of ingested active substance or composition. That is, the approaches described herein can deliver active substances to olfactory and/or taste receptors, producing physiological benefit (e.g., up-regulating and, or down-regulating human metabolism), while delivering almost no active substance to the gastro-intestinal (GI) tract. Second, for the purposes of delivery solely to the nose, as in the delivery of decongestants for the common cold or other respiratory dysfunction, the approaches described herein can produce greater physiological effect per nominal dose delivered to the nose than any other approach known by applicants, as in a spray or respiration from the environment into the nose.

Other kinds of water aerosols that are delivered to the mouth include electronic cigarettes and a methodology known as “Le Whaf.” Electronic cigarettes nebulize material into the mouth however with particle sizes that are small enough to penetrate the lungs and via the act of respiration, not sipping. Le Whaf produces mean particles sizes that are larger than 50 microns, thus not optimally suited to penetration into the nose. Other kinds of retro-nasal delivery of active substances exist in the form of highly volatile or aromatic lozenges, or food and drink, as in chocolate cake or a cup of coffee. These latter all involve ingested material with nominal masses placed in the mouth of greater than 1 gram.

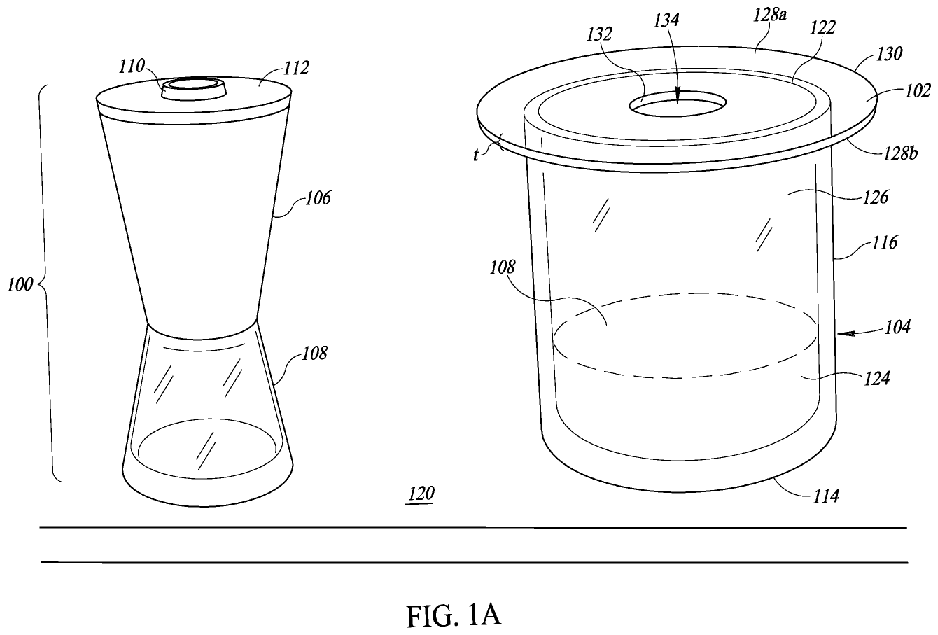

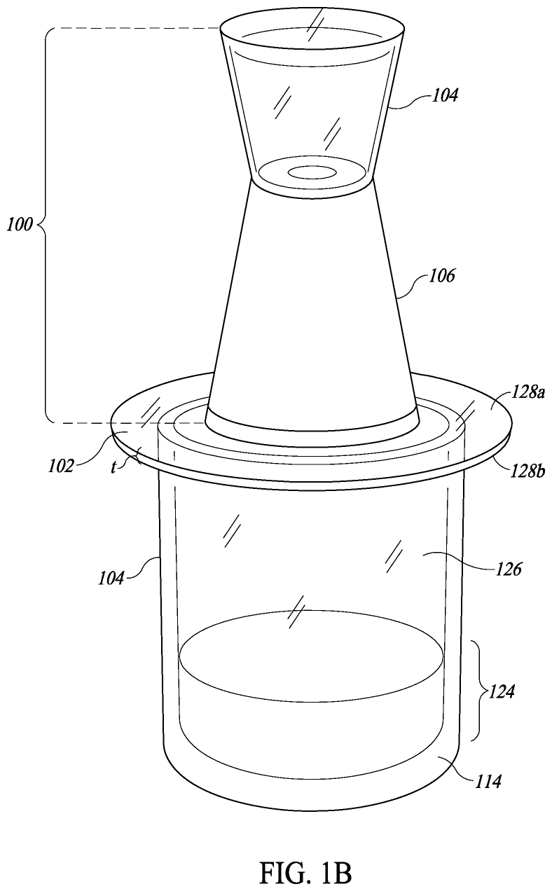

FIG. 1A shows a dispenser 100, interface 102 and vessel 104 according to at least one illustrated embodiment, the interface 102 shown positioned over an opening 106 of the vessel 104 and the dispenser 100 shown spaced from the interface 102 and vessel 104.

As described in more detail herein, the dispenser 100 includes a body 106 and a media reservoir 108. The media reservoir 108 may serve as a base of the dispenser 100 at a bottom end thereof. The dispenser 100 may have a nozzle or outlet 110 positioned at a top end thereof, via which media is dispensed, for example in a vapor or aerosol form. The dispenser 100 may also include a mating surface at the top end, for example illustrated as a mating disk 112.

In some implementations, the media reservoir 108 may implement a fluid reservoir and may be comprised of a polymer, elastomer, or other light-weight, durable material that may be used to hold a liquid. The media reservoir 108 may be formed of one or more plastics, for example an ABS or polycarbonate plastic. The plastic may be injection molded or vacuum molded to form the media reservoir 108. The type of material or process employed to form the body 106 from the material should not be considered limiting. In some implementations, the media reservoir 108 may include an interior cavity that forms the media reservoir 108 that may be used to hold and contain one or more substances as a fluid or other material (e.g., powder, gel, colloidal suspension) that carries scent media (e.g., scent molecules) and/or active substances (e.g., therapeutics). In some implementations, for example, the media reservoir 108 may be sized and dimensioned to hold up to 100 mL of the fluid 110. In some implementations, the media reservoir 108 may be sized and dimensioned to hold a maximum amount of the fluid that is less than 100 mL (e.g., 5 mL, 10 mL, 20 mL, 40 mL, or 50 mL). The substance or media may be any liquid or other material that is, or that carries, the scent, therapeutics or other substance(s) that are released when the substance or media transitions to a vapor or aerosol and is released into the interior 118 of the vessel 104. The dispenser 100 may include an aperture, nozzle or outlet 110 that forms part of the fluidly communicative substance path for the substance to be transferred from the media reservoir 108 to the interior 118 of the vessel 104, for example as the vapor or aerosol 126. The vapor or aerosol 126 may advantageously comprise readily-soluble water droplets have a median size range of approximately 2 microns to approximately 50, 20, or 10 microns. Thus, the readily-soluble water droplets are too large for significant penetration into the lungs, while being small enough to be carried into the nose. The vapor or aerosol 126 may result in a physiological response from some users when those users encounter the active substance(s) transported by the vapor or aerosol 126.

The dispenser 100 may include two separable parts (e.g., body 106, reservoir 108), with a seam therebetween, for example to allow a power source to be replaced or parts serviced. The parts may be include threads or other coupling structures (not shown) allowing the parts to be screwed apart and screwed together. One or more gaskets (not shown) can provide hermetical sealing of the interior of the dispenser 100 from an exterior thereof.

The vessel 104 can take any of a large variety of forms, for instance drinking glasses, mugs, and/or other containers. The vessel 104 typically has a base 114, a peripheral wall 116 extending from the base 114 that delimits an interior 118 of the vessel 104 from an exterior 120 thereof. The vessel 104 typically has an opening 122 at a top thereof to allow the ingress of material (e.g., edible material) and egress of material (e.g., edible material 124, media 126). The vessel 104 may be comprised of a solid material, such as a hard plastic, ceramics, glass, or other similar material.

The interface 102 may take the form of a substrate or slab or plate. The interface 102 may have a first major face 128a and a second major face 128b, the second major face 128b opposed to the first major face 128a across a thickness t of the interface 102. The interface 102 may, for example take the form of an annular disc or plate, having an outer circumference 130 and an inner circumference 132 that provides a through-hole or port 134. The outer circumference 130, as dictated by a corresponding radius or diameter should be sufficiently large to completely cover the opening 122 of the vessel 104. The inner circumference or port 132 should be sized to receive at least a portion of the nozzle or outlet 110 of the dispenser 100. While the interface 102 is illustrated in FIGS. 1A and 1B as a distinct and separate part from the dispenser 100, in some implementations the interface 100 can be attached (e.g., fixed, removably detachably coupled) to the dispenser 100.

FIG. 1 B shows the dispenser 100, interface 102 and vessel 104 of FIG. 1A, the interface 102 shown positioned over the opening 122 of the vessel 104 with the second major face 128b resting on a rim of the vessel 102, and the dispenser 100 shown inverted and positioned on the first major face 128a of the interface 102 to dispense media 126 into the interior 118 of the vessel 104 while the interface 102 substantially retains the media 126 within the interior 118 of the vessel 104.

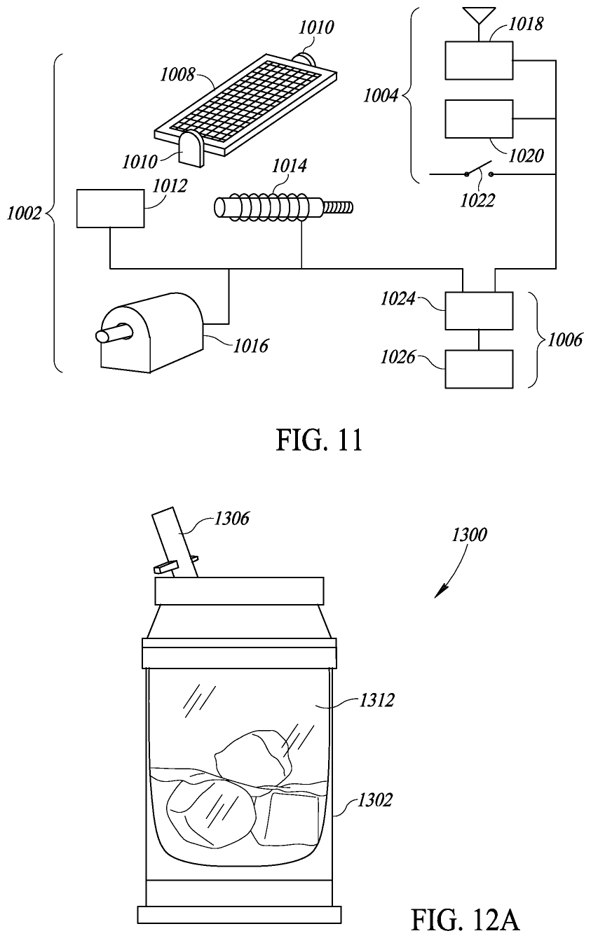

As described in detail below with reference to FIG. 11, the dispenser 100 includes actuator may be used to turn the substance (e.g., fluid) stored in the media reservoir 108 into the vapor or aerosol 126 that is transmitted to the interior 118 of the vessel 104. In some implementations, for example, the actuator is a nebulizer, for instance in the form of a transducer that oscillates a metal mesh to generate a mist, for example held or dispensed from in the interior 118 of the vessel 104. The transducer may oscillate at a frequency of about 175 kHz±5 kHz that is sufficient to atomize the substance or media held in the media reservoir 108. The frequency of oscillation of such a transducer may be increased or decreased depending up on the properties of the substance or media or other materials held within the media reservoir 108. In such an implementation, that transducer may form an annular ring with a metal-mesh included within a center portion of the transducer. As such, the substance or media may be transported to the metal mesh, via, for example, capillary action, where it is atomized into the vapor or aerosol 126 as a result of the oscillation of the transducer. In such an implementation, the actuator may be located towards the bottom of the body 106 such as to be in contact with the substance or media.

In some implementations, the actuator 106 is electrically coupled to a battery that may provide a power source for the oscillation of the scent actuator. The battery may be small and lightweight, such as the batteries used for small electronic devices (e.g., hearing aids). In some implementations, the battery is at least partially embedded within the body 106. In some implementations, the battery is selectively removable and replaceable, such as when the battery can no longer provide sufficient charge to operate the scent actuator. Other types of power sources may be provided, such as a power source comprised of one or more photovoltaic panels and associated components that may convert light into energy that can be used to operate the scent actuator, an array of super- or ultra-capacitor cells, or an array of fuel cells.

The fluidly communicative substance path includes an opening or aperture, nozzle, or outlet 110 through which atomized fluid particles can be introduced to the interior 118 of the vessel 104 as the vapor or aerosol 126. The opening or outlet 110 for the fluidly communicative path may extend across the thickness t of the interface 102 from the first major face 128a to the second major face 128b. In some implementations, the opening, nozzle, or outlet 110 for the fluidly communicative substance path is substantially circular in shape, and is sized and dimensioned to be received through the opening 134 of the interface 102, for example closely received therethrough. In some implementations, opening 134 of the interface 102 may include an outer ring comprised of a compressible, elastic substance that, when compressed, forms a hermetic seal between the interface 102 and a portion of the nozzle or outlet 110. As such, the compressible barrier may prevent any vapor or aerosol from escaping during dispensing or transferring to the interior 118 of the vessel 104.

The dispenser 100, interface 102 and vessel 104 may be provided or vended or packaged as a kit. For example, the kit may include a standard sized dispenser 100, and an interface 102 with at least an outer diameter that exceeds an outer diameter of the vessel 104 or exceeds an inner diameter of the opening 134 of the vessel 104. Alternatively, the dispenser 100 may be provided with a selection of interface of varying dimensions as a kit. For instance, a dispenser 100 may be packaged with a set of four interfaces, each with a larger outer dimension than the other. An end user can then select the appropriate interface 102 for the particular vessel the intend to use.

The interface 102 may for example be made of a polymer, for example acrylic, and may have an outer characteristic dimension of approximately 4 inches, and an inner characteristic dimension of approximately 2 cm, and a thickness of approximately ⅛ inch.

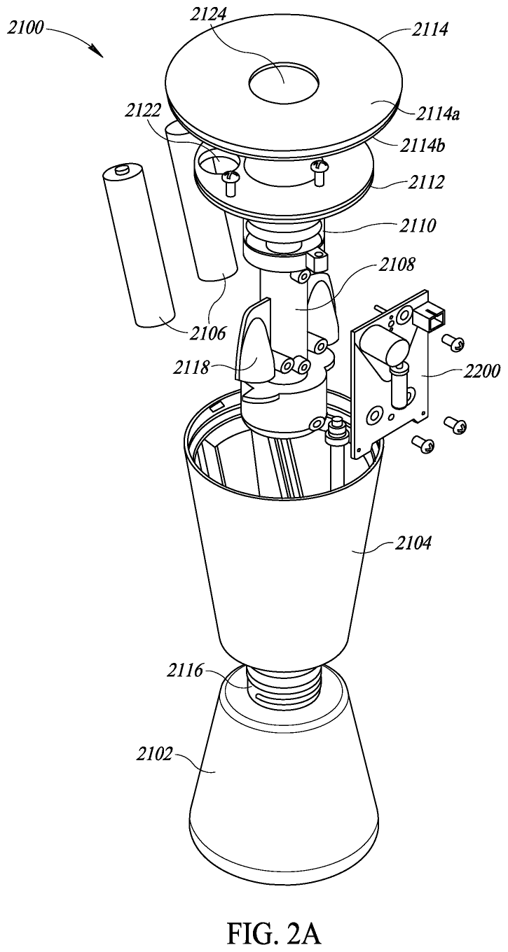

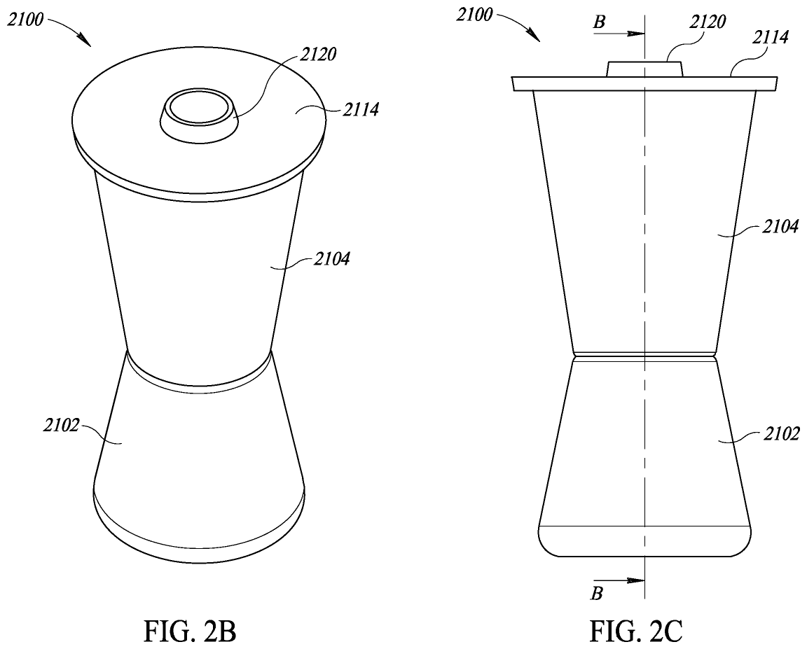

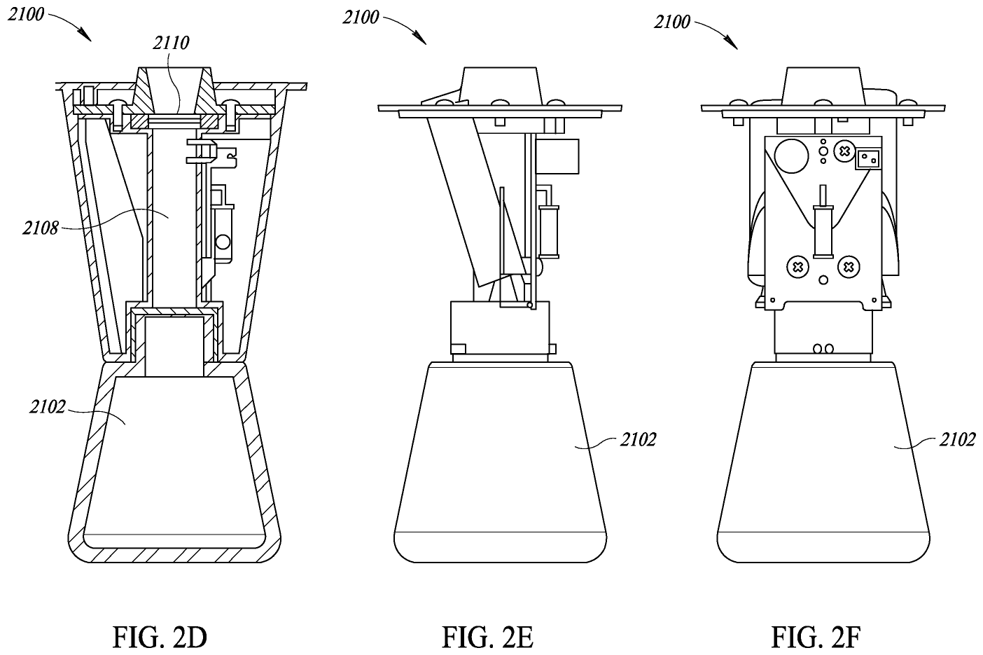

FIGS. 2A-2F illustrate various views of a handheld delivery dispenser 2100 for producing and delivering a cloud of vaporized scent media or scent media in aerosol form. The dispenser 2100 can include any of the features of any of the other dispensers described herein, and can be used in combination with any of the other devices described herein, such as the dispenser 100. As illustrated in FIG. 2A, delivery dispenser 2100 includes a base 2102, which can be transparent and which includes a hollow container or tank or vial, in some cases having a volume or capacity of less than 100 mL, for holding scent media in a liquid form. The base 2102 also includes an upwardly-extending hollow conduit, tube, or pipe 2116, through which the scent media can be poured out of the base 2102 in a liquid form. An exterior surface of the conduit 2116 includes a set of threads.

The delivery dispenser 2100 also includes a top or upper portion or main body 2104, which includes a hollow housing and the electronic and mechanical components of the delivery dispenser 2100. Such components include a printed circuit board 2200 and associated components coupled thereto, a pair of batteries 2106, a hollow conduit, tube, or pipe 2108, a piezo-electric device 2110, which can include or be physically coupled to a mesh screen having a mesh size of 3 microns, of 4 microns, of 6 microns, of 20 microns, or of between 3 and 20 microns, as well as an internal cover 2112, and an external cover 2114, which can be transparent or translucent. The housing of the main body 2104 can be opaque or translucent, and can have a specific color such as red, orange, yellow, green, blue, purple, brown, black, or white. The internal cover 2112 can have an appearance matching that of the housing of the main body 2104. In particular, the internal cover 2112 can be opaque if the housing of the main body 2104 is opaque or translucent if the housing of the main body 2104 is translucent, and can have a specific color matching that of the housing of the main body 2104, such as red, orange, yellow, green, blue, purple, brown, black, or white.

The conduit 2108 includes a relatively wide top end portion, a relatively narrow middle portion and a relatively wide bottom end portion sized to extend around the conduit 2116 of the base 2102. An inner surface of the bottom end portion of the conduit 2108 includes threads complementary to the threads of the conduit 2116 so that the conduits 2108 and 2116 can be threadedly engaged and thereby coupled to one another. When the conduits 2108 and 2116 are coupled to one another, liquid scent media can be poured out of the base 2102 through the conduit 2116 and into the conduit 2108. The relatively wide top end portion of the conduit 2108 is sized and configured to house the piezo-electric device 2110 at the top end of the conduit 2108, so that the liquid scent media can flow through the conduit 2108 from the bottom end portion thereof to the piezo-electric device housed at the top end portion thereof.

The conduit 2108 also includes a pair of flanges 2118 that are coupled to opposing outer side surfaces of the middle portion of the conduit 2108, and that extend laterally outward from the respective side surfaces as well as in a direction aligned with the overall length of the conduit 2108. The flanges 2118 each include a recess or cradle that is shaped and configured to cradle a portion of one of the batteries 2106, to partially restrain the batteries 2106 when the dispenser 2100 is assembled. The internal cover 2112 includes a generally circular or disk-shaped main body portion and a hollow and truncated cone-shaped portion 2120 that extends upward from the main body portion. The main body portion of the internal cover 2112 includes a pair of openings or apertures 2122 that extend through the main body portion. Each of the apertures 2122 is sized and configured to cradle a portion of one of the batteries 2106, to partially restrain the batteries 2106 when the dispenser 2100 is assembled.

The delivery dispenser 2100 includes an integral interface 2114 in the form of a generally circular or disk-shaped main body portion and an opening or aperture 2124 that extends through the main body portion, for example forming an annular plate. The aperture 2124 is sized and configured to fit snugly around a portion of the outer surface of the cone-shaped portion 2120 of the internal cover 2112 when the dispenser 2100 is assembled. The interface 2114 has a lower major face 2114b (FIG. 2A) that is adjacent the internal cover 2112 and an upper major face 2114b that faces away from the remainder of the dispenser 2100, providing a mating surface to mate with an upper edge or rim of a vessel. As such, the interface 2114 should have an outer dimension that is at least, a preferably is larger, than a dimension of an opening of the vessel to which the interface will mate. For example, the interface 2114 may have an outer circumference with a radius or diameter that is at least equal to a radius or diameter of an opening or body of a vessel. While generally illustrated with a circular profile, the vessels and/or interfaces can have other profiles, including rectangular, hexagonal, octagonal or even irregular.

FIGS. 2B, 2C, and 2D illustrate perspective, side, and cross-sectional side views, respectively, of the delivery dispenser 2100. FIGS. 2E and 2F illustrate two different side views of the delivery dispenser 2100 with the housing of the main body 2104 removed to reveal internal components of the main body 2104.

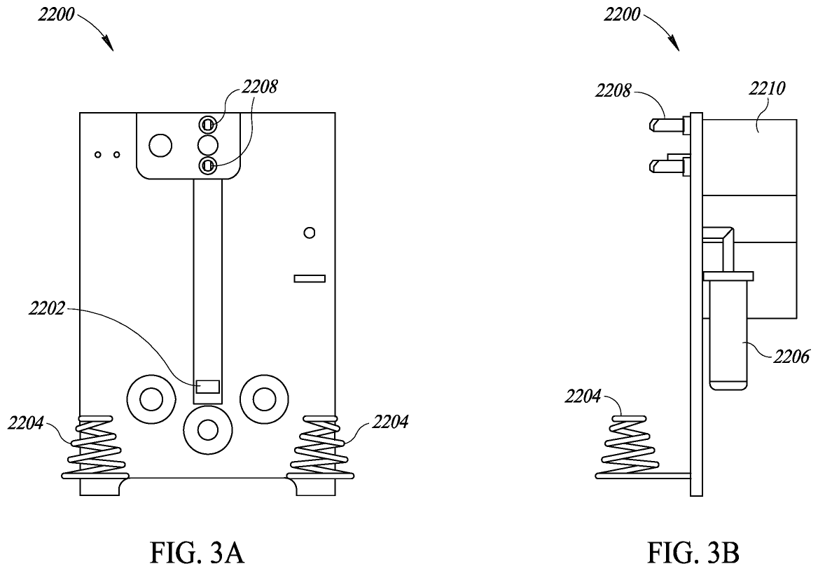

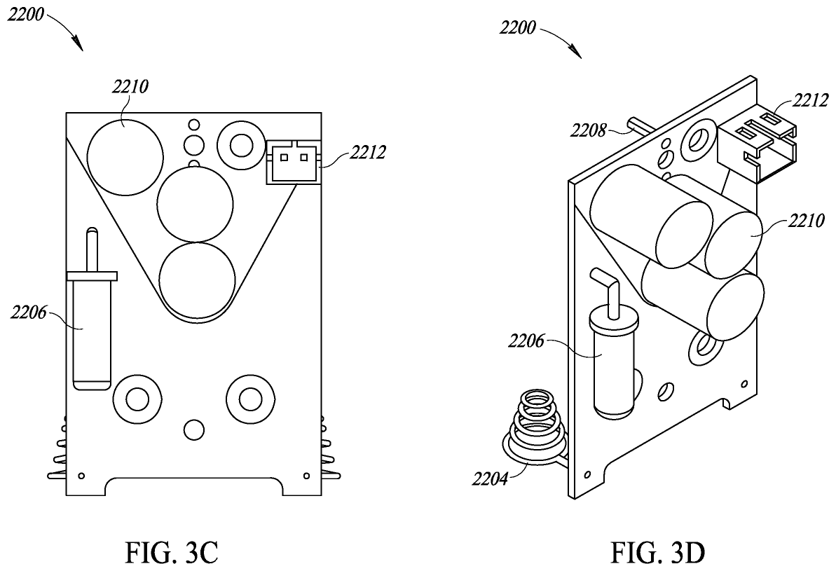

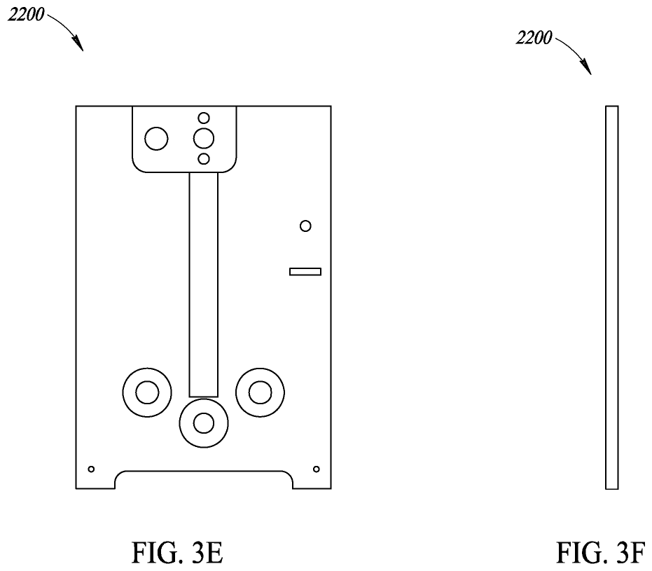

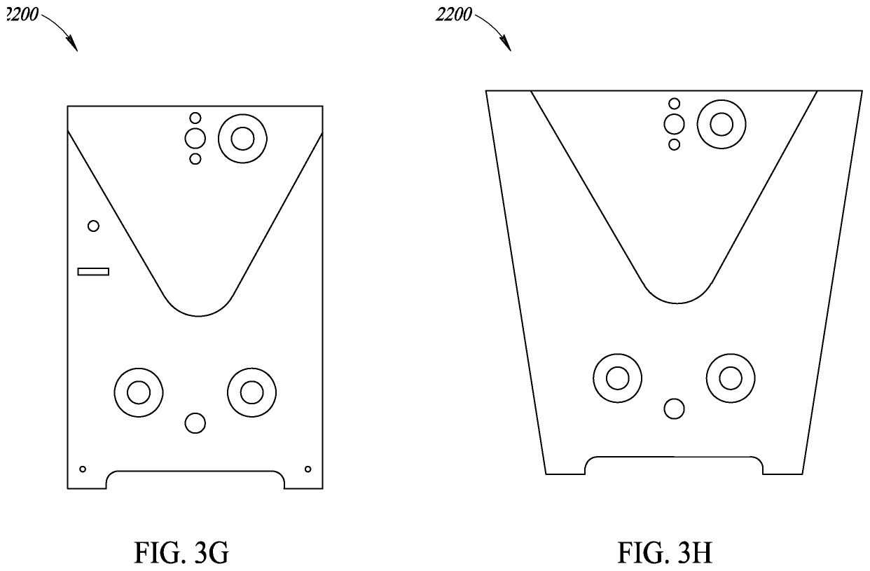

FIGS. 3A-3D illustrate the printed circuit board 2200 of the delivery dispenser 2100 with associated components coupled thereto. FIG. 3A is a rear view of the printed circuit board 2200 and illustrates that the printed circuit board 2200 includes an LED 2202 physically and electrically coupled to the rear surface thereof, which can be operable to light up or turn on when the delivery dispenser 2100 is generating a cloud of vaporized scent media or scent media in aerosol form, and to turn off when the delivery dispenser 2100 is not generating a cloud of vaporized scent media or scent media in aerosol form. The LED can be useful to a user of the dispenser 2100 because when the LED lights up, the user can be confident that power is being supplied to the printed circuit board 2200. FIG. 3A also illustrates that the rear surface of the printed circuit board 2200 is physically and electrically coupled to two metallic springs 2204, each of which is positioned and configured to act as a contact for, and to partially support or cradle, one of the batteries 2106. One of the springs 2204 can act as a positive contact, while the other of the springs 2204 can act as a negative contact, for the batteries 2106, such that the batteries 2016 will be installed within the dispenser 2100 with their polarities reversed with respect to one another.

FIG. 3B is a side view of the printed circuit board 2200 and illustrates that the rear surface of the printed circuit board 2200 is also physically and electrically coupled to a plurality of gold pins 2208 to which a fluid sensor can be physically and electrically coupled. FIG. 3C is a front view of the printed circuit board 2200 and illustrates that the front surface of the printed circuit board 2200 can include an electrical connector 2212, which can be a JST connector, to allow an operator to physically and electrically couple other electronic devices, such as the piezo-electric device 2110, to the printed circuit board 2200 and to allow the printed circuit board and other associated components coupled thereto to communicate with (e.g., transmit signals to or receive signals from) such other electronic devices including the piezo-electric device 2110. FIG. 3C also illustrates that the front surface of the printed circuit board 2200 is physically and electrically coupled to a tilt sensor 2206, which can include an accelerometer or a ball tilt switch in which a ball moves and connects pins to complete an electrical circuit when the dispenser 2100 is tilted or inverted, and to a plurality of capacitors 2210 for storing electrical energy. FIG. 3D is a perspective view of the printed circuit board 2200 and illustrates a perspective view of the printed circuit board 2200 with the associated components coupled thereto. FIGS. 3E-3G illustrate the printed circuit board 2200 without the associated components coupled thereto.

As illustrated in FIGS. 2A, 2D-2F, the rear of the printed circuit board 2200, illustrated directly in FIG. 3A, faces toward the conduit 2108 and the center of the delivery dispenser 2100, while the front of the printed circuit board 2200, illustrated directly in FIG. 3C, faces away from the conduit 2108 and the center of the delivery dispenser 2100. In some implementations, the printed circuit board 2200 receives power from a source at between 2.0 and 3.4 Volts DC, and provides power to a load at 140 KHz and at 65 Volts peak-to-peak. FIG. 3H illustrates a front view of an alternative shape and configuration for the printed circuit board 2200. FIGS. 3A-3H illustrate some examples of possible dimensions of the printed circuit board 22, with the numbers used in millimeters. It will be understood that the specific dimensions provided in these Figures are merely examples of possible suitable dimensions.

To operate the delivery dispenser 2100, a user can fill the base 2102 with media, e.g., in a liquid form, and assemble the dispenser 2100 except for the batteries 2106 and the external cover 2114, such as by screwing or threading the base 2102 onto the main body 2104. The user can then insert the batteries 2106 into the dispenser 2100 through the apertures 2122 in the internal cover 2112, such that the batteries are partially cradled by the recesses of the flanges 2118, and such that bottom terminals of the batteries 2106 are in electrical contact with the springs 2204. The interface 2114 can be removably or fixedly coupled to the rest of the dispenser 2100, such as by threading or press-fitting the external cover into a top end of the main body 2104. An underside of the interface 2114 can include a strip of electrically-conductive material, such as metal, which can engage the top terminals of the batteries 2106 and electrically couple the upper terminal of one of the batteries 2106 to the upper terminal of the other one of the batteries 2106.

The user can then lift and tilt or even invert the dispenser 2100, such that the fluid flows, under the force of gravity, from the base 2102, through the conduit 2108, to the piezo-electric dispenser 2110. Once the user tilts or inverts the dispenser 2100, the tilt sensor 2206 can generate and transmit a signal indicating that the dispenser 2100 has been tilted. Further, once the fluid flows to the piezo-electric device 2110, the fluid may come into contact with a fluid sensor coupled to the pins 2208 and generate and transmit a signal indicating that the fluid has reached the fluid sensor. Further still, the dispenser 2100 can include a pressure-sensitive switch on a bottom surface thereof which, when the dispenser 2100 is picked up off of a flat surface, can generate and transmit a signal that the dispenser 2100 has been picked up. In some embodiments, the dispenser 2100 includes no manually-operated switches or buttons, and receives no input from the user, other than one, two, or three of the signals described above.

Upon receipt of any one, any two, or all three of such signals, the dispenser 2100 can activate the piezo-electric device 2110 to begin generating a cloud of vaporized scent media or scent media in aerosol form from the scent media in liquid form. Because the dispenser 2100 is tilted sideways or upside-down (inverted), the cloud of vaporized scent media or scent media in aerosol form can flow out of the dispenser 2100 through the hollow cone-shaped portion 2120, and can be poured into a container or vessel for subsequent consumption. The container or vessel may, or may not, already contain some edible substance, e.g., a beverage or solid food). In some implementations, the dispenser 2100 includes an internal timer and automatically turns off or de-activates the piezo-electric device 2110 to stop generating the cloud of vaporized scent media or scent media in aerosol form after a time period of about 5, about 10, about 15, or about 20 seconds. In other implementations, the dispenser 2100 continues to operate and generate the vaporized scent media or scent media in aerosol form until the dispenser 2100 is once again oriented upright or placed back on a flat horizontal surface.

When the fluid within the base 2102 runs out, the user can unscrew or unthread of the base 2102 from the main body 2104 of the dispenser 2100, refill the base 2102 with more of a desired scent media in a fluid form, screw or thread the base 2102 back on to the main body 2104, and then resume using the dispenser 2100. When the batteries 2106 die, no longer power the device 2100, and need to be replaced, the user can remove the interface 2114 from the rest of the dispenser 2100, such as by unscrewing, unthreading, or turning the interface 2114 with respect to the rest of the dispenser 2100. The exhausted batteries 2106 within the dispenser 2100 can then be removed and new batteries 2106 can be installed in their place. The user can then re-install the interface 2114 onto the rest of the dispenser 2100 and resume using the dispenser 2100.

In some implementations, the interface 2114, or a surface of the rest of the dispenser 2100 that engages with the interface 2114, includes a detent, and the detent is engaged as the interface 2114 is turned with respect to the rest of the dispenser 2100 just before the interface 2114 is released from the rest of the dispenser 2100. Engagement of the detent can serve as a signal to the user that the interface 2114 is about to be released from the rest of the dispenser 2100. Once the user releases and removes the interface 2114 from the rest of the dispenser 2100, the batteries are disconnected and the dispenser is unable to operate. Thus, the interface 2114 can act as a switch, where removing the interface 2114 from the rest of the dispenser 2100 switches the dispenser 2100 off and engagement of the interface 2114 with the rest of the dispenser 2100 switches the dispenser 2100 on.

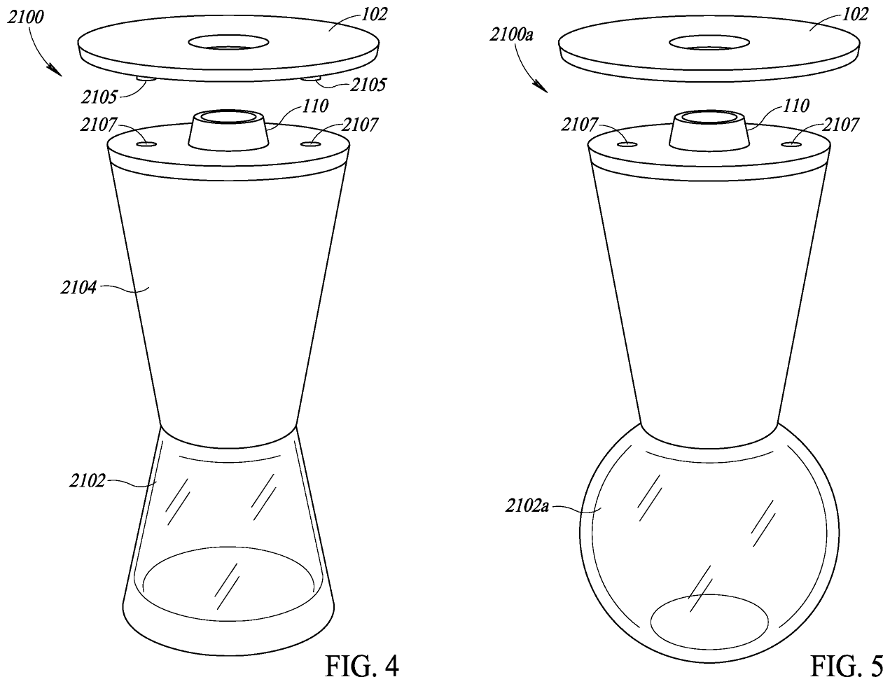

FIG. 4 illustrates a three-dimensional rendering of the dispenser 2100, showing its overall shape. In particular, the base 2102 of the dispenser 2100 has a geometric shape including a truncated cone, and the main body 2104 of the dispenser 2100 has a geometric shape including an inverted truncated cone, such that the dispenser 2100 has an overall geometric shape resembling an hourglass, with a bottom portion that is smaller than its top portion. The interface 102 is shown as a distinct separate element from the dispenser 2100. The interface 102 may simply sit on top of the dispenser 2100, support via a top or inner cover of the dispenser 2100. Alternatively, the interface 102 may be removably coupled to the dispenser 2100 for example via one more magnets. In some implementations, the interface 102 may have one or more magnets 2105 and the dispenser 2100 may include one or more magnets 2107, positioned to magnetically mate with the magnets of the interface 102. Alternatively, as shown in FIG. 5, the interface 102 may be formed of a ferrous metal, and the dispenser 2100 may include one or more magnets 2107 to magnetically mate with the ferrous metal interface 102. Alternatively, other detachable couplings can be used, including tabs and slots, bayonet mounts, or hook and loop fastener.

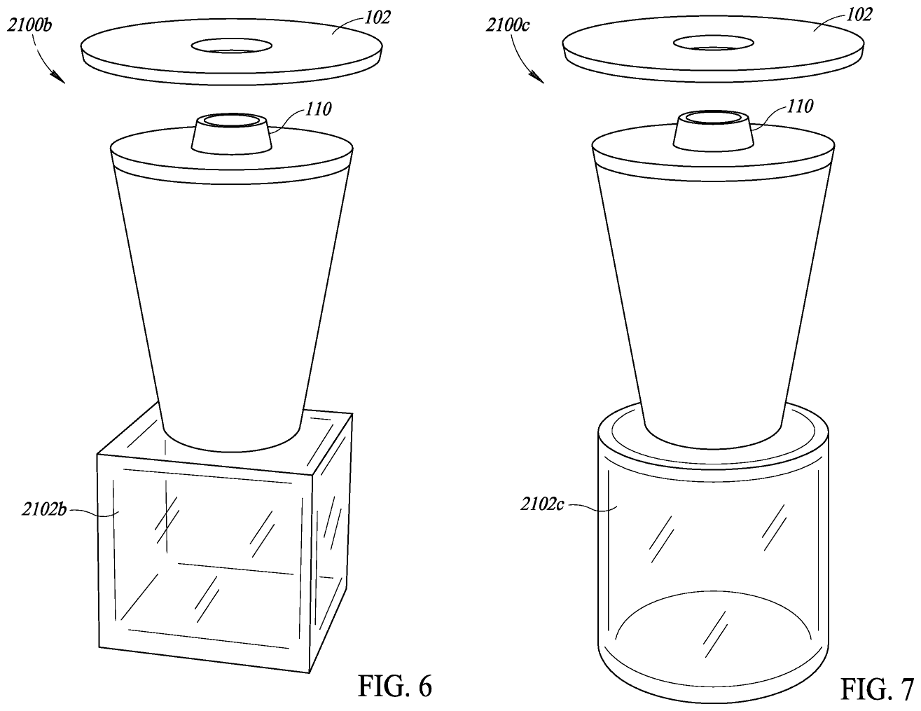

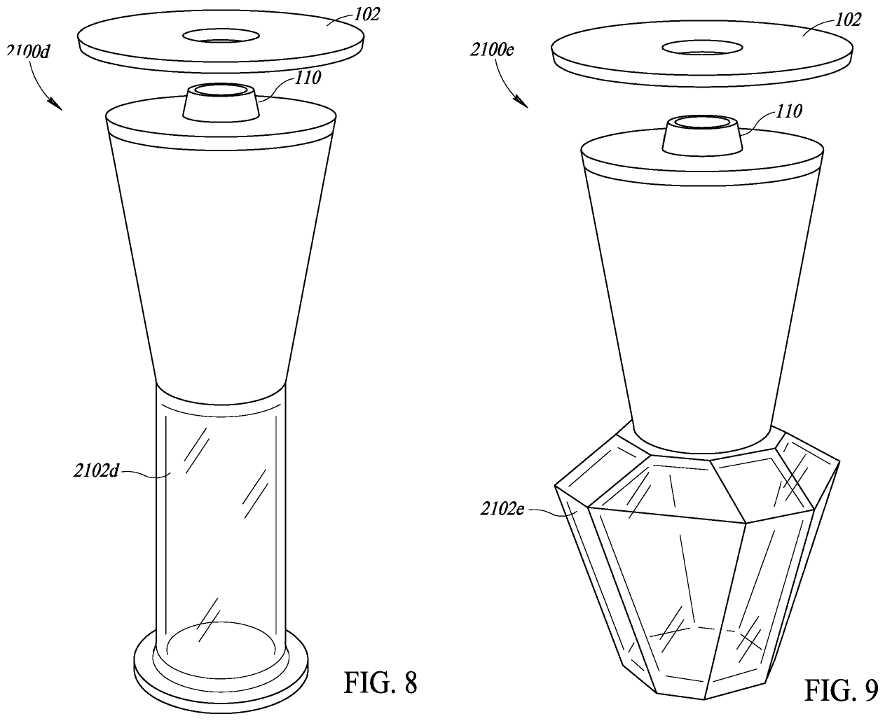

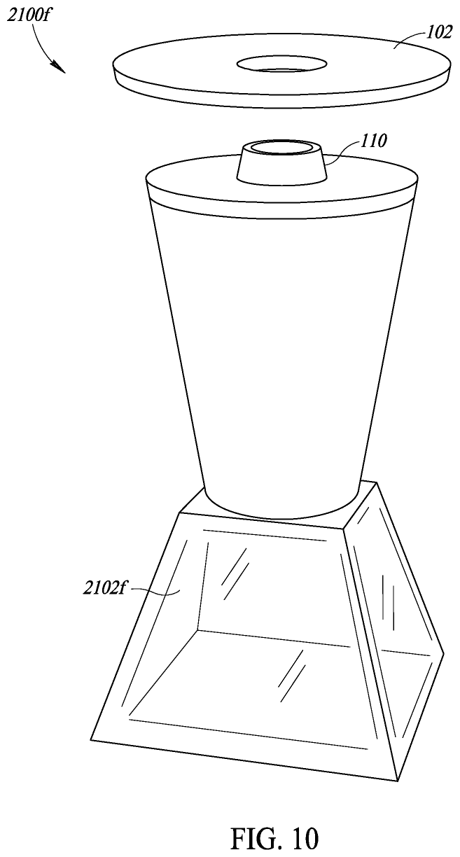

FIG. 5 illustrates one alternative implementation of the dispenser 2100a, in which the base 2102a has a geometric shape including a sphere with a truncated bottom end and a truncated top end. FIG. 6 illustrates another alternative implementation of the dispenser 2100b, in which the base 2102b has a geometric shape including a cube. FIG. 7 illustrates another alternative implementation of the dispenser 2100c, in which the base 2102c has a geometric shape including a relatively short and wide cylinder. FIG. 8 illustrates another alternative implementation of the dispenser 2100d, in which the base 2102d has a geometric shape including a relatively tall and narrow cylinder. FIG. 9 illustrates another alternative implementation of the dispenser 2100e, in which the base 2102e has a geometric shape resembling a truncated diamond, such as resembling a truncated asscher-cut diamond. FIG. 10 illustrates another alternative implementation of the dispenser 2100f, in which the base 2102f has a geometric shape including a truncated four-sided pyramid.

FIG. 11 is an isometric view of a portion of a dispenser, including a nebulizer 1002, one or more actuators 1004, and a control subsystem 1006 and, or other electronics, according to at least one illustrated implementation.

The nebulizer 1002 can include one or more screens 1008 which is supported by a frame 1010 for movement, for example for oscillation or rotation The nebulizer 1002 can include one or more of a piezo-electric element 1012, solenoid 1014 or electric motor 1016 physically coupled to move the screen(s) 1008. The nebulizer 1002 may comprise a screen and a piezo-electric element, solenoid, or an electric motor physically (e.g., mechanically, magnetically) coupled to move (e.g., oscillate, rotate) the screen and thereby cause dispersion of the scent media in the interior of the vessel, for instance as a spray. The nebulizer may, for example, oscillate the screen at ultrasonic frequencies to cause a dispersion of the scent media.

The actuators 1004 may include one or more of transducers or sensors 1020 and, or, switches 1022 communicatively coupled to the control subsystem 1006.

The control subsystem 1006 may, for example, include one or more microcontrollers 1024, microprocessors, field programmable gate arrays, and, or application specific integrated circuits. The control subsystem 1006 may, for example, include one or more nontransitory storage media 1026 that stores at least one of processor-executable instructions or data, which when executed by the microcontroller 1024 causes the microcontroller 1024 to control operation of the dispenser 900, for example in response to one or more inputs. For example, the microcontroller may receive signals from one or more of radios 1018, transducers or sensors 1020 and, or, switches 1022, and control operation of the nebulizer 1002 in response to same. For instance, the control subsystem 1006 may cause the nebulizer to dispense or disperse scent media in response to a first input, and to stop the nebulizer from dispensing or dispersing scent media in response to a second input. Input can include user manipulation of a switch, positioning or orientation of the vessel by the user, or wireless commands from a radio or remote controller.

In some implementations, a control system 1006 may include a microcontroller. A suitable microcontroller may take the form of an 8-bit microcontroller with in-system programmable flash memory, such as the microcontroller commercially available from Atmel Corporation under designation ATMEGA48/88/168-AU. The microcontroller executes a program stored in its memory, and sends signals to control the various other components, such as, for example, the valves. Control signals may, for instance be pulse width modulated (PWM) control signal, particularly where controlling an active power supply device. Otherwise, control signals may take on any of a large variety of forms. For instance, the microcontroller may valves or the actuator 106 simply by completing a circuit that powers the respective value or actuator 106.

The dispenser 100 may optionally include a visual indicator 162 to indicate when the delivery system 100a is operating or turned ON. Although illustrated as a single light emitting diode (LED), the visual indicator 162 may take any of a large variety of forms. The LED may be capable of emitting one, two or more distinct colors. The visual indicator 162 may also indicate other information or conditions, for instance the visual indicator 162 may flash in response to an occurrence of an error condition. A pattern of flashes (e.g., number of sequential flashes, color of flashes, number and color of sequential flashes) may be used to indicate which of a number of possible error conditions has occurred.

The dispenser 100 may include one or more switches or sensors communicatively coupled to operate the nebulizer. The device may, for example, include a switch that is operable from an exterior of the device, for instance a contact switch, a momentary contact switch, a rocker switch, etc. In at least one implementation, the switch may be mechanically coupled to a base or other portion of the device or vessel, and activated manually, e.g., by twisting the base or some part of the vessel. The device may, for example, include one or more sensors, for instance a one-, two- or three-axis accelerometer, a PIR motion sensor, an inductive sensor, a capacitive sensor, and, or Reed switches. The sensor(s) can detect a motion (e.g., upward movement, downward movement) and, or orientation (e.g., tilting, upright) of the device or vessel, and operate the nebulizer in response to some defined motion or orientation. The sensor(s) may detect a touch by the user or other contact (e.g., contact or absence of contact between a base of the device or vessel and some object, for instance a table) and operate the nebulizer in response to some defined touch or contact. The sensor(s) may detect a twisting of a vessel relative to a base, or even the placement of the vessel on the base, and operate the nebulizer in response. The sensor(s) may detect a presence or absence, for example a presence or absence of the vessel with respect to the base or a defined location on the base, and operate the nebulizer in response to some defined presence or absence. For instance, the base or the vessel may include one or more magnets, and the other one of the base or vessel may include a sensor (e.g., a Reed switch) that is response to the presence or absence of a magnet and associated magnetic field in the proximity of the sensor. In at least one implementation, placement of the vessel on the base is detected by the sensor, which provides a signal that causes the nebulizer to begin dispensing or dispersing scent in the interior of the vessel. Removal of the vessel from the base is also detected by the sensor, which in response provides a signal that causes the nebulizer to cease dispensing or dispersing scent in the interior of the vessel.

The control subsystem 1006 may include a timer, for example to operate the nebulizer for a defined time period in response to receiving a control signal from the switch on activation of the switch. Such may be particularly useful for controlling a length of time, and hence and amount of active media dispensed. Such may be particularly useful where the switch is a momentary switch, which, for example, provides a single signal in response to a single activation. The control subsystem 1006 may prevent subsequent dispersal for a defined period of time, or may prevent such until the delivery device has been removed from the vessel and reinsert. The dispenser 100 may include one or more inertial sensors or fluid sensor to detect whether or not the delivery device has been removed from the food or beverage prior to allowing a subsequent dispersal and provide signals indicative of such to the control subsystem 1006. The control subsystem 1006 may monitor a quantity of scent media residing the in the media reservoir 108 or a pressure, for example via one or more pressure sensors, load cells or other sensors. The control subsystem 1006 may cause presentation of a message when the amount remaining media in the media reservoir 108 falls below a threshold level.

The dispenser 100 may include a transducer communicatively coupled to operate the nebulizer. The transducer may, for example, include one or more radios (e.g., cellular transceiver, WI-FI transceiver, Bluetooth transceiver) which receives wireless signals for instance RF or microwave signals for one or more wireless communications devices (e.g., smartphones) or remote controllers. The transducer may, for example, include one or more receivers, for instance an infrared receiver that receivers infrared light signals from a remote controller.

Activation may be synchronized with the delivery of audio, video, or audiovisual media. For example, a smartphone or digital assistance (e.g., Amazon Alexa®, Google Home®, Apple HomePod®) can cause activation of flavorful droplets inside a vessel that a consumer can experience in coordination with the delivery or experience of other digital media, e.g., music, film, video games, virtual reality (VR), augmented reality (AR), etc.

It may be desirable to create food and beverage experiences where the aroma associated with the food or beverage is enhanced, or other than it naturally would otherwise be. In this respect, flavor shapes appetite and cravings. Human appetites and cravings are largely shaped by flavor images that form in the brain while eating and drinking. It is possible to generate these images via delivery of flavorful droplets of minor mass, thereby shaping appetite and cravings without ingestion. Shaping the flavors, independently of the food and beverages themselves, makes it possible to help people eat and drink better and have more pleasurable experiences. This approach can be characterized as an “augmented reality” of scent and flavor, and the associated device denominated as an Augment Reality Vessel for Flavor Perception. The spray of droplets, aerosol or cloud of scent material may be characterized as a scent garnish. Droplets may have a media size of around 500 microns. These droplets may be delivered into open air within a vessel, preferably at angles that permit the droplets or cloud to remain principally contained inside the vessel. The approach can be used in entertainment (e.g., food or cooking programming), and in healthcare (e.g., control of metabolism).

The dispenser 100 includes one or more power sources, for example one or more of: primary battery cells, secondary battery cells, super- or ultra-capacitor cells, and, or fuel cells. The power source(s) may be rechargeable, for instance via a set of electrical contacts or inductive charger element.

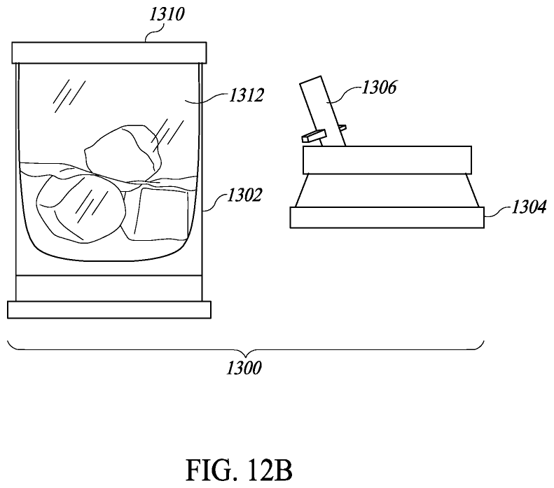

FIGS. 12A and 12B are side elevational views of a dispenser 1300 including a vessel 1302 with a cover 1304, and a conduit or straw 1306, according to at least one illustrated implementation. In particular, FIG. 12A illustrates the cover 1304 is positioned to close a top or opening 1310 (FIG. 12B) of the vessel 1302, while FIG. 12B the cover 1304 removed from the vessel 1302 to open the top or opening 1310, thereby providing unhindered access to an interior 1312 of the vessel 1302.

In operation, the cover 1304 may be sufficient to ensure that a spray or aerosol dispersed or dispensed by a dispenser at least initially remains in the interior 1312 of the vessel 1302. Alternatively or additionally, the nebulizer or a port thereof may optionally be positioned and oriented to dispense or disperse media in a direction that at least initially substantial retains the dispensed or dispersed media within an interior 1312 of the vessel 1302.

The vessel 1302 may contain a liquid (e.g., illustrated as a beverage) and, or a solid (e.g., food; illustrated as ice cubes). The media may be dispensed or dispersed (e.g., spray of droplets, aerosol) over the liquid and, or, solid which the user will imbibe, for example via a straw, the media changing or enhancing the liquid or solid which the user will imbibe.

The conduit or straw 1306 that provides a fluidly communicative path through the cover 1304 for a user to imbibe the contents of the vessel 1302 imbued with the substance (e.g., scent, therapeutics).

In some implementations, the scent media used in any one of the delivery devices described herein can include functional additives or active agents, in some cases for therapeutic applications, such as vitamins, minerals, supplements, other nutrients to provide nutrition, or drugs, medications, or pharmaceutical agents. As specific examples, such compounds can be used to assist a user in recovering from addictions such as opiate or food addictions, or to assist a user in controlling their metabolism. In some cases, such compounds can be provided in a liquid, and either a concentrated or a purified form, and can be vaporized or aerosolized by one of the delivery devices described herein and poured over a food or a beverage that either does not contain such compounds or that contains such compounds in a much lower concentration than the vapor or aerosol.

As one specific example, a commercial syrup, such as a commercial cinnamon syrup, such as is commercially available under the brand name Torani, can be diluted with water in a ratio of between one third and one half syrup to between one half and two thirds water, and then mixed with a water-soluble vitamin such as vitamin B12 such that the vitamin makes up about five percent of the mass of the mixture. The mixture can then be poured into a vial within one of the delivery devices described herein, and the dispenser or delivery device can be operated to vaporize or aerosolize the mixture and create a cloud of the vapor or aerosol over a food or a beverage such as a glass half-full of water. In such an implementation, the mass of the cloud over the water is around 1 milligram and the cloud contains about 50 micrograms of the vitamin. In this way, a user, such as a patient, can consume a daily dose of vitamin B12 by consuming ten sips of water if with each sip, the user consumes the cloud of vapor or aerosol over the water and then pours a new cloud of vapor or aerosol for consumption with the next sip of water.

As another example, the same process can be performed but with a peppermint syrup and with vitamin D mixed into the syrup and water such that the vitamin makes up about one percent of the mass of the mixture. In such an implementation, the cloud of vapor or aerosol over the water contains about 10 micrograms of the vitamin. In this way, a user, such as a patient, can consume a daily dose of vitamin D by consuming two sips of water if with each sip, the user consumes the cloud of vapor or aerosol over the water and then pours a new cloud of vapor or aerosol for consumption with the next sip of water.

As another example, the same process can be performed but with a salty caramel syrup and with folic acid mixed into the syrup and water such that the folic acid makes up about five percent of the mass of the mixture. In such an implementation, the cloud of vapor or aerosol over the water contains about 50 micrograms of folic acid. In this way, a user, such as a patient, can consume a daily dose of folic acid by consuming eight sips of water if with each sip, the user consumes the cloud of vapor aerosol over the water and then pours a new cloud of vapor or aerosol for consumption with the next sip of water.

This process can be adjusted for use with a wide variety of vitamins, minerals, other nutrients, or medications, including to deliver a 700-900 μg dose of Vitamin A (RAE), a 300 μg dose of Biotin, a 80 μg dose of Vitamin K, a 120 μg dose of Chromium, a 150 μg dose of Iodine, a 75 μg dose of Molybdenum, or a 70 μg dose of Selenium to a user such as a patient. This process can be used to create clouds of vaporized or of droplets of aerosolized compounds where the total mass of the droplets in the cloud is, or is about, or is less than or equal to, 1 milligram.

In at least some implementations, the active substance may provoke a physical response, and may even take the form of a therapeutic. For example, the media may take the form of therapeutic and other substances in a form suitable for retro-nasal delivery, for instance active substance media deliverable as an aerosol comprising readily-soluble droplets have a median size range of approximately 2 microns to approximately 10 microns and comprising one or more active substances, which may be used to modulate human or other animal biological processes or treat medical conditions, and related compositions, as described in International Patent Application PCT/US2018/050250, filed Sep. 10, 2018. For example, the media may take the form of compositions of food, therapeutic and/or other substances in a form suitable for ortho-nasal and retro-nasal delivery, which may be used to modulate human or other animal metabolic processes or treat medical conditions, and related formulations, compositions, and related systems, methods, and articles of manufacture, as described in International Patent Application PCT/US2019/049541, filed Sep. 4, 2019. Such may, for instance include agonists or antagonists of at least one type of transient receptor potential vanilloid (TRPV) receptor, and a sufficient amount of water or a mixture of water and alcohol to aerosolize the composition. The agonists or antagonists may be agonists or antagonists at least one of TRPV1 receptors, TRPV3 receptors, TRPV4 receptors, or TRPV8 receptors. The composition may include two or more types of TRPV antagonists. The composition may include two or more types of TRPV antagonists. The composition may include agonists or antagonists of all: at least one type of TRPV receptors, olfactory receptors, and taste receptors. The composition may include one or more of the following: sodium iodide, sodium chloride, magnesium chloride, capsaicin, and piperine. The composition may include one or more of the following: sodium iodide, sodium chloride, potassium chloride, magnesium chloride, capsaicin, and piperine, and/or combinations of the same. The composition may include one or more of the following: ground cinnamon, a cannabinoid, pimento, onion, clove, thyme, ginger, menthol, Irish cream, lemon, lime, mango, raspberry, watermelon, blueberry, strawberry, popcorn, meat, Resolvin D2 or nicotine. The composition may include linalool. The aerosolized composition may include droplets less than 100 microns in size which carry the agonists or antagonists. A concentration of all agonists or antagonists in the composition may be in a range of 50 micrograms/milliliters to 2 milligrams/milliliter or 1 milligrams/milliliter. As noted, the composition may be delivered ortho-nasally and/or retro-nasally as an aerosol. For any of these, a period over which a substance is dispensed may be calculated to provide a suitable dosage of the given substance to realize a defined or desired therapeutic effect. Thus, a duration may be determined based on a concentration of substance in a volume, taking into account how much of that substance would be effective delivered to the subject (e.g., ratio of substance on volume to that is absorbed or taken in by the body), and a defined or desired dosage for the subject (e.g., based on the type of substance, the desired effect, and/or a weight or other physical attribute of the subject).

U.S. provisional patent application Nos. 62/652,069, filed Apr. 3, 2018, 62/628,395, filed Feb. 9, 2018, and 62/556,974, filed Sep. 11, 2017; 62/687,970, filed Jun. 21, 2018; and 62/838,740; as well as International patent application publication WO 2019/051403 and International patent application PCT/US20/27691 filed Apr. 10, 2020, are hereby incorporated by reference, in their entireties. The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system for use with a vessel having an opening with at least a first characteristic dimension, the system comprising:
a dispenser having a body, an outlet, a reservoir that at least in use holds media to be dispensed, and an actuator controllably operable on the media to cause the media to be dispensed via the outlet;
an interface having a first major face, a second major face opposed to the first major face across a thickness of the interface, at least one of the first or the second major faces having an outer dimension that is at least equal to the first characteristic dimension of the opening of the vessel, and the interface having a through-hole aligned or alignable with the outlet of the dispenser when the dispenser is positioned on one of the first or the second major faces and the other of the first or the second major faces is positioned on the vessel.

2. The system of claim 1 wherein the actuator is controllably operable to cause formation of an aerosol comprising readily-soluble droplets have a median size range of approximately 2 microns to approximately 10 microns and comprising the one or more substances.

3. The system of claim 1 wherein the interface is removably coupled to the body of the dispenser.

4. The system of claim 1 wherein the interface is fixed to the body of the dispenser.

5. The system of claim 1 wherein the interface integral to the dispenser.

6. The system of claim 1 wherein the interface is an annular plate having an outer perimeter and an inner perimeter, the outer perimeter having a radius at least equal to a radius of an opening of the vessel and the outer perimeter greater than a corresponding outer perimeter of the body of the dispenser.

7. The system of claim 1 wherein the interface is an annular plate having an outer perimeter and an inner perimeter, the outer perimeter having a radius at least equal to a radius of a sidewall of the vessel and the radius of the outer perimeter greater than a corresponding radius of an outer perimeter of the body of the dispenser.

8. The system of claim 1 wherein the interface is an annular plate having an outer perimeter and an inner perimeter, the outer perimeter having a radius greater than a radius of a sidewall of the vessel.

9. The system of claim 1 wherein the interface is an annular plate having an outer perimeter and an inner perimeter, the inner perimeter having a radius closely receives a portion of the outlet of the dispenser.

10. The system of claim 1 wherein the outlet of the dispenser is a frusto-conical and the through-hole of the interface closely receives a portion of the outlet of the dispenser.

11. The system of claim 1, further comprising:
a control subsystem communicatively coupled to control the actuator.

12. The system of claim 11, further comprising:
at least one of a switch or a sensor communicatively coupled to the control subsystem and responsive to a position or orientation of the vessel and operable to produce a signal that causes the control subsystem to operate the actuator according to the orientation of the vessel.

13. (canceled)

14. (canceled)

15. A kit, the kit comprising:
a vessel having a base, a side wall and an opening at a top of the vessel, the base and side walls delimiting an interior of the vessel from an exterior thereof, at least one the side wall or opening of the vessel having at least a first characteristic dimension;
a dispenser having a body, an outlet, a reservoir that at least in use holds media to be dispensed, and an actuator controllably operable on the media to cause the media to be dispensed via the outlet;
an interface having a first major face, a second major face opposed to the first major face across a thickness of the interface, at least one of the first or the second major faces having an outer dimension that is at least equal to the first characteristic dimension of at least one of the side wall or the opening of the vessel, and the interface having a through-hole aligned or alignable with the outlet of the dispenser when the dispenser is positioned on one of the first or the second major faces and the other of the first or the second major faces is positioned on the vessel.

16. The kit of claim 15 wherein the actuator is controllably operable to cause formation of an aerosol comprising readily-soluble droplets have a median size range of approximately 2 microns to approximately 10 microns and comprising the one or more substances.

17. The kit of claim 15 wherein the interface is removably coupled to the body of the dispenser.

18. The kit of claim 15 wherein the interface is fixed to the body of the dispenser.

19. The kit of claim 15 wherein the interface integral to the dispenser.

20. The kit of claim 15 wherein the interface is an annular plate having an outer perimeter and an inner perimeter, the outer perimeter having a radius at least equal to a radius of an opening of the vessel.

21. The kit of claim 15 wherein the interface is an annular plate having an outer perimeter and an inner perimeter, the outer perimeter having a radius at least equal to a radius of a sidewall of the vessel.

22. The system of claim 1 wherein the interface is an annular plate having an outer perimeter and an inner perimeter, the outer perimeter having a radius greater than a radius of a sidewall of the vessel.

23. The kit of claim 15 wherein the interface is an annular plate having an outer perimeter and an inner perimeter, the inner perimeter having a radius closely receives a portion of the outlet of the dispenser.

24. The kit of claim 15 wherein the outlet of the dispenser is a frusto-conical and the through-hole of the interface closely receives a portion of the outlet of the dispenser.

25. The kit of claim 15, further comprising:
a control subsystem communicatively coupled to control the actuator.

26. The kit of claim 25, further comprising:
at least one of a switch or a sensor communicatively coupled to the control subsystem and responsive to a position or orientation of the vessel and operable to produce a signal that causes the control subsystem to operate the actuator according to the orientation of the vessel.

27. The kit of claim 15, further comprising:
a hermetically sealed package;
a dosage of an active substance contained in the hermetically sealed package.
